(19) United States
(12) Patent Application Publication
(10) Pub. No.: US 2025/0366937 A1
(43) Pub. Date: Dec. 4, 2025

(54) SYSTEM FOR IMPLANTING WIRE ELECTRODE AND METHOD FOR OPERATING SAME

(71) Applicant: SHANGHAI STAIRMED TECHNOLOGY CO., LTD., Shanghai (CN)

(72) Inventors: Zhengtuo ZHAO, Shanghai (CN); Xue LI, Shanghai (CN); Yang ZHOU, Shanghai (CN)

(21) Appl. No.: 18/876,321

(22) PCT Filed: Jun. 29, 2022
(86) PCT No.: PCT/CN2022/102394

§ 371 Date: Dec. 18, 2024

(30) Foreign Application Priority Data
Jun. 20, 2022 (CN) 202210697119.3

(51) Int. Cl. A61B 34/30; A61B 17/00; A61B 17/34; A61B 34/10; A61B 90/00; A61B 90/30

(52) U.S. Cl. CPC A61B 34/30; A61B 17/3403; A61B 34/10; A61B 90/30; A61B 90/361; A61B 2017/00022; A61B 2017/00398; A61B 2017/00477; A61B 2017/00544; A61B 2034/107

(57) ABSTRACT

A system for implanting an electrode wire (1) is configured for implanting the electrode wire (1) into a brain of an organism, and includes an implantation apparatus (100), a first light source (410), a second light source (420), a first camera (430), a second camera (440), a brain surface camera (450), and a processing apparatus, wherein the implantation apparatus is provided with an implantation movement mechanism that is configured to enable the implantation apparatus (100) to implant the electrode wire (1) at different angles in different orientations.

### TECHNICAL FIELD

The present disclosure relates to a system for implanting a wire electrode, a system for implanting a bioelectrode to a target object, a system for implanting a wire electrode to a target object, and a method for operating the system according to the present disclosure.

### BACKGROUND

A flexible neural electrode, as an important tool in the field of brain-machine interfaces, is currently being developed and gradually improved by scientific research and commercial institutions at home and abroad. Compared with a traditional hard neural electrode, the flexible neural electrode has greatest advantages of excellent mechanical compatibility and difficulty in scar formation in brain tissue. However, due to low mechanical strength and easy deformation of the wire electrode itself, it is prone to break in the process of implantation into the brain tissue, making it difficult to penetrate mater into the tissue. Therefore, the tool and manner for implanting the flexible neural electrode currently are technical difficulties.

The currently existing implantation solutions are mainly divided into two classes: manual implantation and machine-assisted implantation. The manual implantation is to implant wire electrodes by a pure manual operation, and generally includes steps of, first, preparing all wire electrodes to be implanted and guide needles corresponding to the respective wire electrodes in advance, with the need to fix the wire electrodes onto the respective guide needles by means of an adhesive or ferrule in advance, and then finding a brain tissue area to be implanted into, and implanting a row of guide needles with the wire electrodes into the target area simultaneously. In this method, many problems occur, including: 1) long preparation time; 2) one implantation can only implant one row of a limited number of wire electrodes due to limitation by an overall size of the electrode, so that if a number of wire electrodes needed to be implanted exceeds one row of wire electrodes, a plurality of combinations of guide needles plus wire electrodes need to be prepared, which consumes a large amount of preparation time and resources; 3) no way to freely select a site for implantation due to the need of simultaneous implantation of the one row of wire electrodes every time; 4) insufficient implantation speed, which may result in the guide needles failing to penetrate dura mater to implant the wire electrodes.

In summary, there are many problems in the manual implantation, and in view of these problems, robot-assisted implantation is currently the most promising solution. Compared to a human being, a robot is able to do work more accurately and is able to do repetitive work consistently. At present, there are still many problems, for example, an implantation head is mounted on a three-axis moving stage, which results in that the implantation head cannot perform implantation at different angles and can only perform rectilinear motion on XYZ axes. Since a surface of a human brain is not a plane, an implantation angle cannot be predicted for the wire electrode. Another problem is that, the implantation head in a form of needle plus clip easily causes the wire electrode to break, unless the hardness of the wire electrode is high enough, but the high hardness will cause increased damage to brain tissue, so that the structure has a risk.

### SUMMARY

An objective of the present disclosure is to provide a system for implanting a wire electrode, a system for implanting a bioelectrode to a target object, a system for implanting a wire electrode to a target object, and a method for operating the system according to the present disclosure, which can overcome a deficiency in the prior art.

According to a first aspect of the present disclosure, there is provided a system for implanting a wire electrode, the system configured for implanting the wire electrode into a brain of an organism, the system including: an implantation apparatus including an implantation needle configured to engage, with a head portion thereof, a free end of the wire electrode, so as to take the wire electrode to move, an implantation feed mechanism configured to move the implantation needle along a longitudinal direction of the implantation apparatus, and an implantation actuation mechanism configured to drive the implantation needle so that the head portion of the implantation needle penetrates into the brain; a first light source for providing first light suitable for observing the implantation needle and the free end of the wire electrode; a second light source for providing second light suitable for observing a surface of the brain; a first camera configured to acquire a first image of the head portion and the free end of the wire electrode with assistance of the first light; a second camera configured to acquire a second image of the head portion and the free end of the wire electrode with assistance of the first light, wherein an optical path of the first camera and an optical path of the second camera are angled with respect to each other; a brain surface camera configured to acquire a third image of the surface of the brain with assistance of the second light; and a processing apparatus configured to identify a relative position of the head portion and the free end of the wire electrode based on the first image and the second image, and identify an implantable area of the surface of the brain based on the third image, wherein the implantation apparatus is provided with an implantation movement mechanism configured to enable the implantation apparatus to implant the wire electrode at different angles in different orientations.

According to a second aspect of the present disclosure, there is provided a system for implanting a bioelectrode to a target object, the system including: an actuation mechanism configured to drive a guide apparatus engaged with the bioelectrode to move, so that the guide apparatus, together with the bioelectrode, enters the target object; a position adjustment mechanism configured to adjust a position of the actuation mechanism, so that the guide apparatus approaches a target implantation area of the target object; d an orientation adjustment mechanism configured to adjust an orientation of the actuation mechanism, so that the guide apparatus is capable of entering, at the target implantation area, the target object at a specific angle with respect to a surface of the target implantation area.

According to a third aspect of the present disclosure, there is provided a system for implanting a wire electrode to a target object, the system including an implantation subsystem including: an actuation mechanism configured to drive a guide apparatus engaged with a wire electrode to move in a direction of approaching a target object at a first speed along a longitudinal direction of the actuation mechanism, so that the guide apparatus, together with the wire electrode, enters the target object; and a longitudinal position adjustment mechanism configured to adjust a position of the actuation mechanism at a second speed along the longitudinal direction, so that the guide apparatus approaches a target implantation area of the target object along the longitudinal direction, wherein the first speed is greater than the second speed.

According to a fourth aspect of the present disclosure, there is provided a method for operating the system according to the present disclosure, wherein the methods includes: arranging the wire electrode on a wire electrode holder; identifying, by the processing apparatus, a relative position between the free end of the wire electrode and the head portion of the implantation needle based on the first image and the second image, and controlling the implantation apparatus and/or the wire electrode holder to move based on the relative position, so that the head portion of the implantation needle is aligned with the free end of the wire electrode along the longitudinal direction; moving, by the implantation feed mechanism, the implantation needle along the longitudinal direction of the implantation apparatus for engagement with the free end of the wire electrode, and disengaging the wire electrode from the wire electrode holder; identifying, by the processing apparatus, an implantable area of the brain based on the third image, and determining a target position for implantation in the implantable area; controlling, by the processing apparatus, the implantation apparatus to move, so that the head portion of the implantation needle is aligned with the target position implantation feed mechanism at a specific angle; causing, by the implantation feed mechanism implantation feed mechanism, the implantation needle to take the wire electrode to move to a predetermined distance from a surface at the target position; and driving, by the implantation actuation mechanism, the implantation needle together with the wire electrode to move forwards.

### BRIEF DESCRIPTION OF THE DRAWINGS

The accompanying drawings, which constitute a part of this specification, illustrate embodiments of the present disclosure and, together with the description, serve to explain the principles of the present disclosure.

FIGS. 1 to 3 are schematic perspective views, from different perspectives, of a system for implanting a wire electrode according to an embodiment of the present disclosure.

FIG. 4 is a schematic view of an implantation apparatus or actuation mechanism as well as an arc-shaped rail for adjusting an orientation of the implantation apparatus or actuation mechanism according to an embodiment of the present disclosure.

FIG. 5 is a detailed schematic perspective view of an implantation apparatus or actuation mechanism according to an embodiment of the present disclosure.

FIG. 6 is a side view of the implantation apparatus or actuation mechanism of FIG. 5.

FIG. 7 is a detailed schematic perspective view of an implantation needle and a second implantation carrier for fixing the implantation needle of FIG. 5.

FIGS. 8A to 8E are schematic views of steps of wire electrode guiding and spraying in a method for operating the system according to the present disclosure, according to an embodiment of the present disclosure.

FIG. 9 is a schematic view of a spatial arrangement of vision modules according to an embodiment of the present disclosure.

Note that in the embodiments described below, the same reference numeral is sometimes used in common among different drawings to denote the same part or parts having the same function, and a repetitive description thereof will be omitted. In some cases, similar items are indicated using similar reference numbers and letters, and thus, once a certain item is defined in one drawing, it need not be discussed further in subsequent drawings.

For ease of understanding, positions, dimensions, ranges, and the like of the structures shown in the drawings and the like sometimes do not necessarily indicate actual positions, dimensions, ranges, and the like. Therefore, the present disclosure is not limited to the positions, dimensions, ranges, and the like disclosed in the drawings and the like.

### DETAILED DESCRIPTION

The present disclosure will be described below with reference to the accompanying drawings, which illustrate several embodiments of the present disclosure. It should be understood, however, that the present disclosure may be presented in many different ways and is not limited to the embodiments described below; in fact, the embodiments described below are intended to make the disclosure of the present disclosure more complete, and to fully convey the scope of protection of the present disclosure to those skilled in the art. It should also be understood that the embodiments disclosed herein can be combined in various ways to provide more additional embodiments.

It should be understood that the terminology used herein is only for describing specific embodiments, and is not intended to limit the scope of the present disclosure. All terms (including technical and scientific terms) used herein have meanings commonly understood by those skilled in the art, unless otherwise defined. Well-known functions or structures may not be described in detail for brevity and/or clarity.

Herein, when it is described that an element is located “on”, “attached” to, “connected” to, “coupled” to, or “contacted” with another element, and so on, the element can be directly located on, attached to, connected to, coupled to, or contacted with the other element, or an intermediate element can be present. In contrast, when it is described that an element is “directly located on”, “directly attached to”, “directly connected to”, “directly coupled to”, or “directly contacted with” another element, no intermediate element will be present. Herein, a feature arranged “adjacent” to another feature, can refer to the feature having a portion overlapped with the adjacent feature or a portion located above or below the adjacent feature.

Herein, elements or nodes or features “coupled” together may be mentioned. Unless expressly stated otherwise, “couple” means that an element/node/feature may be mechanically, electrically, logically, or otherwise connected to another element/node/feature directly or indirectly to allow interaction, even though the two features may not be directly connected. That is, “couple” is intended to include both direct and indirect connections of elements or other features, including a connection using one or more intermediate elements.

Herein, a spatial relationship term, such as “above”, “below”, “left”, “right”, “front”, “back”, “upper”, or “lower”, may describe a relationship between a feature and another feature in a drawing. It should be understood that the spatial relationship term includes different orientations of a device in use or operation in addition to an orientation shown in the drawing. For example, when the device in the drawing is turned upside down, a feature originally described as “below” another feature may now be described as “above” the other feature at this time. The device may also be otherwise oriented (rotated 90 degrees or at other orientations), and at this time, a relative spatial relationship will be interpreted accordingly.

Herein, the term “A or B” includes “A and B” and “A or B”, rather than exclusively including only “A” or including only “B”, unless specifically stated otherwise.

Herein, the term “exemplary” means “serving as an example, instance, or illustration”, and not as a “model” that is to be reproduced exactly. Any implementation exemplarily described herein is not necessarily to be construed as preferred or advantageous over other implementations. Moreover, the present disclosure is not limited by any expressed or implied theory presented in the above TECHNICAL FIELD, BACKGROUND, SUMMARY, or DETAILED DESCRIPTION.

Herein, the term “substantially” means encompassing any minor variations imperfections in design caused by or manufacturing, tolerances of devices or components, environmental effects and/or other factors. The term “substantially” also allows for differences from a perfect or ideal situation caused by parasitic effect, noise, and other practical considerations that may exist in a practical implementation.

In addition, for reference purposes only, similar terms such as “first” and “second” can also be used herein, and thus are not intended to be limiting. For example, unless clearly indicated by the context, the terms “first”, “second” and other such numerical terms involving structures or elements do not imply a sequence or order.

It should further understood that the term be “comprise/include”, when used herein, specifies the presence of stated features, steps, operations, units and/or components, but do not preclude the presence or addition of one or more other features, steps, operations, units and/or components, and/or combinations thereof.

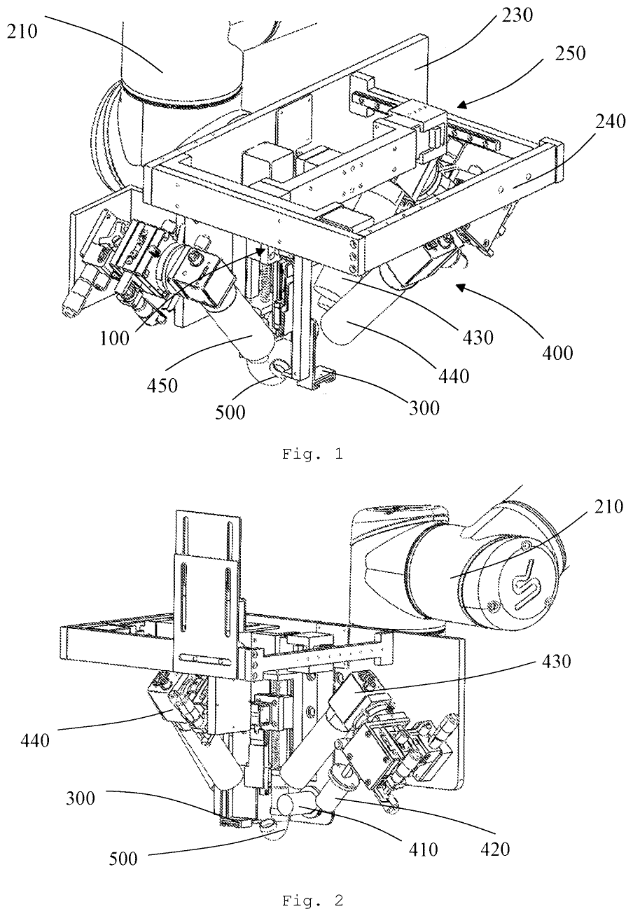

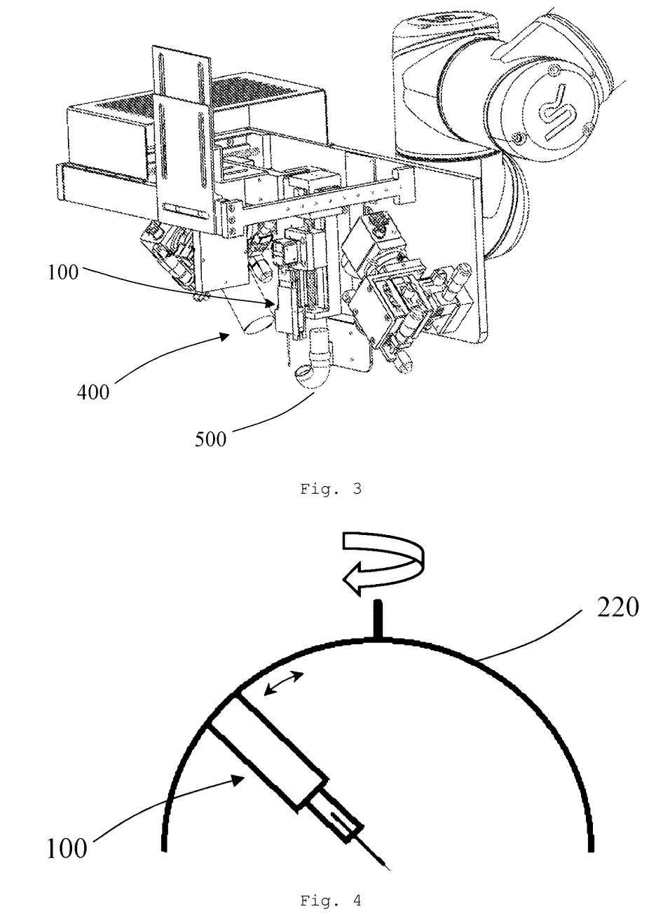

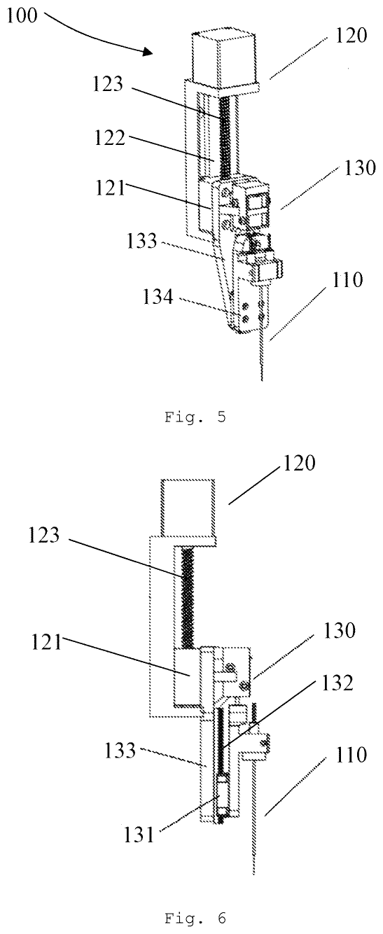

FIGS. 1 to 3 show schematic perspective views, from different perspectives, of a system for implanting a wire electrode 1 according to an embodiment of the present disclosure. FIG. 4 shows a schematic view of an implantation apparatus 100 or actuation mechanism as well as an arc-shaped rail 220 for adjusting an orientation of the implantation apparatus 100 or actuation mechanism according to an embodiment of the present disclosure. FIG. 5 shows a detailed schematic perspective view of the implantation apparatus 100 or actuation mechanism according to an embodiment of the present disclosure. FIG. 6 shows a side view of the implantation apparatus 100 or actuation mechanism of FIG. 5. FIG. 7 shows a detailed schematic perspective view of an implantation needle 110 and a second implantation carrier 134 for fixing the implantation needle 110 of FIG. 5.

As shown, the system for implanting the wire electrode 1 according to the present disclosure includes an implantation apparatus 100. The implantation apparatus 100 may include an implantation needle 110, an implantation feed mechanism 120, and an implantation actuation mechanism 130. The implantation needle 110 may be configured to engage, with a head portion 111 thereof, a free end of the wire electrode 1, so as to take the wire electrode 1 to move. The implantation feed mechanism 120 may be configured to move the implantation needle 110 along a longitudinal direction of the implantation apparatus 100. The implantation actuation mechanism 130 may be configured to drive the implantation needle 110 so that the head portion 111 of the implantation needle 110 penetrates into a brain. In addition, the system for implanting the wire electrode 1 further includes an implantation movement mechanism, which is provided with the implantation apparatus 100. The implantation movement mechanism may be configured to enable the implantation apparatus 100 to implant the wire electrode 1 at different angles in different orientations. In other words, a system for implanting a bioelectrode to a target object according to the present disclosure includes an actuation mechanism configured to drive a guide apparatus engaged with the bioelectrode to move, so that the guide apparatus, together with the bioelectrode, enters the target object. The guide apparatus may be configured as the implantation needle 110. The target object may include the brain. The target object may include a non-flat surface. For example, the target object may be a brain surface. The bioelectrode may include a flexible wire electrode 1 (also referred to as a wire-like flexible electrode). The system for implanting a bioelectrode to a target object further includes a position adjustment mechanism configured to adjust a position of the actuation mechanism so that the guide apparatus approaches a target implantation area of the target object, and an orientation adjustment mechanism configured to adjust an orientation of the actuation mechanism so that the guide apparatus is capable of entering, at the target implantation area, the target object at a specific angle with respect to a surface of the target implantation area.

As shown in FIGS. 1 to 3, the implantation movement mechanism includes a robotic arm 210 that is freely movable in space. The robotic arm 210 may be a conventional multi-axis robotic arm 210. With the robotic arm 210, 6 degrees of freedom or 7 degrees of freedom can be implemented. As shown in FIG. 4, the implantation movement mechanism may further include an arc-shaped rail 220 on which the implantation apparatus 100 can slide. On the arc-shaped rail 220, a groove not shown may be provided. On an end of the implantation apparatus 100 away from the implantation needle 110, a slider matched with the groove is provided, by which the implantation apparatus 100 can slide along a arc-shaped track on the arc-shaped rail 220. The arc-shaped rail 220 is rotatable about a rotation axis normal to an arc of the arc-shaped rail 220 and extending through a center of the arc-shaped rail 220. Thus, by combining the rotation of the arc-shaped rail 220 with the sliding of the implantation apparatus 100 on the arc-shaped rail 220, the implantation apparatus 100 can be substantially oriented in any orientation within a hemispherical area. Here, at least one of the robotic arm 210 or the arc-shaped rail 220 may be provided. In FIGS. 1 to 3, an embodiment in which only the robotic arm 210 is provided is shown. It should be appreciated by those skilled in the art that in other embodiments, only the arc-shaped rail 220 may be provided, or the robotic arm 210 and the arc-shaped rail 220 may be provided simultaneously. When the robotic arm 210 and the arc-shaped rail 220 are provided simultaneously, the implantation apparatus 100 is connectable with the robotic arm 210 via the arc-shaped rail 220. By directly arranging the implantation apparatus 100 on the arc-shaped rail 220, an that generates a motion involving angular changes can, from the perspective of error propagation, be made closer to the execution terminal, i.e. the implantation needle 110, so that an angular change error can be effectively controlled.

At least a part of the position adjustment mechanism may be implemented as the robotic arm 210. The position adjustment mechanism may include a coarse position adjustment module and a fine position adjustment module. At least a part of the coarse position adjustment module and the orientation adjustment mechanism may be implemented as the robotic arm 210. Thus, with the robotic arm 210, on the one hand, translation in various directions or coarse position adjustment of the entire assembly arranged on the robotic arm 210 and the guide apparatus belonging to the assembly can be implemented, and on the other hand, adjustment of the orientation of the guide apparatus can be implemented. The fine position adjustment module may include a micro motor. The micro motor may be arranged at an end of the guide apparatus or implantation needle 110 that is close to the target object. For example, the head portion 111 of the implantation needle 110 may be equipped with a micro motor capable of moving the head portion 111 in a transverse direction of the implantation apparatus 100. Thus, after the coarse position adjustment and orientation adjustment by the robotic arm 210, fine adjustment on the position of the guide apparatus can be performed by the micro motor on the guide apparatus according to specific situations of the target object, to perform the guidance and implantation operations with a precise position. For the adjustment of the orientation, the orientation adjustment mechanism may further include an arc-shaped rail 220, a first end of the actuation mechanism away from the guide apparatus being mounted to the arc-shaped rail 220, so that the orientation adjustment mechanism adjusts the orientation of the actuation mechanism. The arc-shaped rail 220 can rotate about its rotation axis as described above. The orientation adjustment mechanism, like the arc-shaped rail 220, may be provided on the position adjustment mechanism, so that the position adjustment mechanism adjusts the position of the actuation mechanism by adjusting the position of the orientation adjustment mechanism. In the embodiments shown in FIGS. 1 to 3, the actuation mechanism is fixedly arranged on a back plate 230 which is mounted on the robotic arm 210. The system for implanting the wire electrode 1 includes a wire electrode 1 holder, which is capable of holding the wire electrode 1 and arranged on a carrying frame 240 fixed on the back plate 230. The wire electrode 1 holder is movably arranged on the carrying frame 240. The wire electrode 1 holder is connected to the carrying frame 240 by a plane movement mechanism which is configured to move along both a direction substantially parallel to the back plate 230 and a direction substantially perpendicular to the back plate 230. In other words, an electrode fixation apparatus 300 for fixing the electrode may be arranged on a transverse adjustment module 250, so that the electrode fixation apparatus 300 may move along a transverse direction, and the transverse adjustment module 250 may move on a longitudinal adjustment module, whereby the electrode fixation apparatus 300 as a whole can move in the transverse and longitudinal directions. In this case, the actuation mechanism is fixed in the frame structure mounted on the robotic arm 210, while the electrode fixation apparatus 300 for fixing the electrode is movable to adjust a relative position between the actuation mechanism and the electrode. In other embodiments, the position adjustment mechanism may further include a longitudinal adjustment module and a transverse adjustment module 250, wherein the longitudinal adjustment module is configured to adjust the position of the actuation mechanism in the longitudinal direction of the actuation mechanism, so that the guide apparatus approaches the target implantation area in the longitudinal direction; and the transverse adjustment module 250 is configured to adjust the position of the actuation mechanism in a plane perpendicular to the longitudinal direction, so that the guide apparatus approaches the target implantation area in the transverse direction. The actuation mechanism may be arranged on the longitudinal adjustment module, which may move on the transverse adjustment module 250, or the actuation mechanism may be arranged on the transverse adjustment module 250, which may move the longitudinal adjustment module. In this case, the actuation mechanism is movable in the frame structure, while the electrode fixation apparatus 300 for fixing the electrode can be fixed. By arranging the actuation mechanism on the longitudinal adjustment module or the transverse adjustment module 250, sufficient mounting space can be provided for mounting of the arc-shaped rail 220. Of course, when the actuation mechanism is fixedly arranged on the frame structure, in order to provide mounting space for the mounting of the arc-shaped rail 220, it is possible to extend perpendicularly to the back plate 230, for example, a fixing member, on which the arc-shaped rail 220 may be rotatably mounted. The transverse adjustment module 250 and/or the longitudinal adjustment module may be configured as a motor, such as a stepper motor.

In the embodiment shown in FIGS. 1 to 3, the head portion 111 of the implantation apparatus 100 is arranged on a sliding plate which can move quickly on a sliding rail by the implantation actuation mechanism 130 to perform the implantation operation. Another embodiment of the implantation apparatus 100 is shown in FIGS. 5 to 7. In the other embodiment, the implantation apparatus 100 may further include a first implantation carrier 133 for arranging the implantation actuation mechanism 130, which is capable of performing rectilinear motion by the implantation feed mechanism 120. The implantation feed mechanism 120 may include a first guide block 121 and a first rectilinear rail 122. The first guide block 121 is capable of performing rectilinear motion on the first rectilinear rail 122. The first guide block 121 is capable of performing rectilinear motion on the first rectilinear rail 122 via a drive mechanism, the drive mechanism being capable of transforming rotational motion into rectilinear motion of the first guide block 121. The implantation feed mechanism 120 may be configured as a stepper motor. The drive mechanism may be configured as a screw drive mechanism 123. The first implantation carrier 133 is integrally configured with the first guide block 121 of the implantation feed mechanism 120. The first implantation carrier 133 may also be detachably arranged on the first guide block 121 of the implantation feed mechanism 120. The first implantation carrier 133 can, for example, be connected to the first guide block 121 of the implantation feed mechanism 120 via threaded connection. The implantation apparatus 100 may further include a second implantation carrier 134 for fixing the implantation needle 110, which can be driven by the implantation actuation mechanism 130 to perform rectilinear motion, so that the head portion 111 of the implantation needle 110 penetrates into the brain. The implantation actuation mechanism 130 may be configured as a high-speed driving motor, which drives, by a magnetic force, the second implantation carrier 134 to perform a high-speed implantation operation. The second implantation carrier 134 is equipped with a second guide block 131 and a second rectilinear rail 132, the second guide block 131 being capable of performing rectilinear motion on the second rectilinear rail 132, the second implantation carrier 134 being arranged on the second guide block 131, so that the second implantation carrier 134, when driven by the implantation actuation mechanism 130, performs rectilinear motion on the second rectilinear rail 132 via the second guide block 131 in a guided manner. The implantation actuation mechanism 130 may be configured as an electromagnetic actuation mechanism, which is capable of driving the second implantation carrier 134 together with the second guide block 131 to move on the second rectilinear rail 132 for a defined stroke, so that the head portion 111 is moved for the defined stroke. The implantation actuation mechanism 130 can also be configured as a pneumatic actuation mechanism, with a stopper provided on the second rectilinear rail 132, the pneumatic actuation mechanism being capable of driving the second implantation carrier 134 together with the second guide block 131 to move on the second rectilinear rail 132, until the second guide block 131 is stopped at the stopper, so that the head portion 111 is moved for a defined stroke. The implantation feed mechanism 120 configured as a stepper motor and the driving screw may move the entire implantation actuation mechanism 130 downwards to a specified height, and then the implantation needle 110 is driven by the implantation actuation mechanism 130 to perform the implantation of the electrode. After the implantation is completed, the implantation needle 110 is returned upwards to an initial height by the implantation feed mechanism 120.

It can be clearly seen from FIG. 7 that the second implantation carrier 134 can include an implantation needle fixation block 135, on which the implantation needle 110 can be detachably fixed. The implantation needle 110 may be configured in two pieces, wherein the implantation needle 110 may include a tube portion 112 and the head portion 111, and the head portion 111 can be fixed on an end of the tube portion 112 or embedded into the tube portion 112. The implantation needle 110 can be fixed on the implantation needle fixation block 135 via an adapter, wherein an end of the tube portion 112 away from the head portion 111 can be fixed on the implantation needle fixation block 135 via the adapter in a manner of threaded fixation or epoxy fixation. The implantation needle 110 can be connected on the adapter in a manner of material locking, wherein the adapter and the tube portion 112 can be connected, for example, by adhesion, welding or the like.

For the penetration of the head portion 111 into the brain, the implantation actuation mechanism 130 can make the head portion 111 penetrate into the brain at a speed between 0.5 m/s and 5 m/s or between 0.01 m/s and 10 m/s. In an example, before implantation on brain nerve, a target object for implantation needs to be anesthetized; after scalp is prepared and cut to expose cranium, the cranium is opened in the target implantation area to form a brain window; and then dura mater within the brain window is uncovered to expose a brain surface. The implantation actuation mechanism 130 may drive the head portion 111 to move towards the target implantation area at a speed greater than or equal to 1 m/s, so that the head portion 111 can puncture pia mater into the target implantation area. In an example, instead of uncovering the dura mater within the brain window, stereotactic dural puncture may be alternatively performed at a target site for the implantation, e.g., by using pulsed field ablation (PFA), thermal ablation, microwave ablation, light wave ablation, radio frequency ablation, laser ablation, cryoablation, and other ablation means. Preferably, the pulsed field ablation is used. This is because the pulsed field ablation is non-contact, non-thermal, specific ablation compared to other ablation means. Then, the implantation actuation mechanism 130 can drive the head portion 111 to move towards the opened implantation site for the implantation. A speed of the movement may be equal to or preferably slightly higher than the speed of the movement of the head portion 111 with the dura mater uncovered. In an example, instead of using the above two manners, an outer table (a part of cranium that is compact bone) of the cranium of the target object for implantation may be alternatively ground away, for example, using a surgical cranial drill, but the brain surface is not exposed. The implantation actuation mechanism 130 may drive the head portion 111 to move towards the target implantation area at a speed greater than or equal to 3 m/s, so that the head portion 111 may puncture the thinned cranium into the target implantation area.

In the implantation, there is a need to control an implantation depth, for which two parameters are involved: an initial implantation position (referring to a position along a z-axis of the implantation apparatus 100) and an implantation stroke. The implantation stroke is, as described above, implemented by the defined stroke generated by the electromagnetic or pneumatic actuation mechanism itself. The initial implantation position may be embodied as a distance between a tip of the head portion 111 and the brain surface. To determine this distance, the system according to the embodiment of the present disclosure may include a brain surface detection apparatus, which is capable of detecting that the conductive head portion 111 is in contact with the brain surface. The brain surface detection apparatus may include a detection circuit, with the head portion 111 connected on the detection circuit and another part of the implanted organism connected to the circuit, wherein a voltage measurement apparatus is connected on the circuit, so that when the head portion 111 is in contact with the brain surface, the circuit is closed and the voltage measurement apparatus measures a voltage to indicate the head portion 111 contacting the brain surface. After the head portion 111 is in contact with the brain surface, the implantation feed mechanism 120 is controlled so that the implantation needle 110 retracts by a predetermined distance, and thus the tip of the head portion 111 of the implantation needle 110 is located at the predetermined distance from the brain surface, i.e., at the initial position. The predetermined distance d may be determined from a depth D that is required for the tip of the head portion 111 to enter the target implantation area and the defined stroke S that the electromagnetic/pneumatic actuation mechanism has, i.e. d=S−D. The depth D that is required for the tip of the head portion 111 to enter the target implantation area can be determined from a depth that is required for the electrode to be implanted and a position at which the electrode is engaged with the head portion 111.

In other words, an system for implanting a wire electrode 1 to a target object according to an embodiment of the present disclosure includes an implantation subsystem including: an actuation mechanism configured to drive a guide apparatus engaged with the wire electrode 1 to move in a direction of approaching a target object at a first speed along a longitudinal direction of the actuation mechanism, so that the guide apparatus, together with the wire electrode 1, enters the target object; and a longitudinal position adjustment mechanism configured to adjust a position of the actuation mechanism at a second speed along the longitudinal direction, so that the guide apparatus approaches a target implantation area of the target object along the longitudinal direction, wherein the first speed is greater than the second speed. The actuation mechanism is further configured to drive the guide apparatus that enters the target object to retract in a direction away from the target object at a third speed along the longitudinal direction, so that the guide apparatus exits from the target object. Here, the first speed and the third speed are both between 0.5 m/s and 5 m/s, or between 0.01 m/s and 10 m/s. As described above, different speeds may be set as needed to implement pia mater puncture, dura mater puncture, and thin cranium puncture. An acceleration of the retraction of the guide apparatus driven by the actuation mechanism is between 25 m/s² and 35 m/s²; the retraction of the guide apparatus at a large acceleration facilitates disengagement of the electrode from the guide apparatus, and avoids, if possible, the guide apparatus taking out the electrode when exiting from the target implantation area. The actuation mechanism may include an electromagnetic actuation module, a high-speed motor actuation module, or a pneumatic module. The actuation mechanism is further configured to drive the guide apparatus to move for a specific stroke in the longitudinal direction, and the implantation subsystem further includes: an implantation depth control module configured to control the longitudinal position adjustment mechanism, so that a tip of the guide apparatus is located at a predetermined distance from a surface of the target implantation area, and then control the actuation mechanism to drive the guide apparatus to move. The implantation depth control module is further configured to control the longitudinal position adjustment mechanism, so that the guide apparatus approaches the target implantation area, until the tip of the guide apparatus is in contact with the surface of the target implantation area, and then control the longitudinal position adjustment mechanism, so that the guide apparatus retracts by a predetermined distance, and thus the tip of the guide apparatus is located at the predetermined distance from the surface of the target implantation area. The implantation depth control module detects that the tip of the guide apparatus is in contact with the surface of the target implantation area by a detection circuit that is closed upon the tip of the guide apparatus contacting the surface of the target implantation area.

In addition, the implantation subsystem includes: an orientation adjustment mechanism configured to adjust an orientation of the actuation mechanism, so that the guide apparatus is capable of entering, at the target implantation area of the target object, the target object at a specific angle with respect to the surface of the target implantation area. Similarly as described above, the implantation subsystem further includes: a transverse position adjustment mechanism configured to adjust the position of the actuation mechanism in a plane perpendicular to the longitudinal direction, so that the guide apparatus is aligned with the target implantation area. The implantation subsystem may further include an electrode fixation apparatus 300 configured to detachably fix, on a surface thereof close to the target object, the wire electrode 1. In this embodiment, the guide apparatus is movable, while the electrode fixation apparatus 300 may be stationary. In other embodiments, the electrode fixation apparatus 300 may also be provided with a transverse position adjustment mechanism, so that a position of the electrode fixation apparatus 300 is adjusted in a plane perpendicular to the longitudinal direction, while the position of the actuation mechanism may be stationary. The transverse position adjustment mechanism may have an adjustment accuracy of less than 4 um. In both of the above embodiments, a relative position between the actuation mechanism and the electrode fixation apparatus 300 can be changed. Of course, in other embodiments, both the electrode fixation apparatus 300 and the actuation mechanism may be configured to be movable.

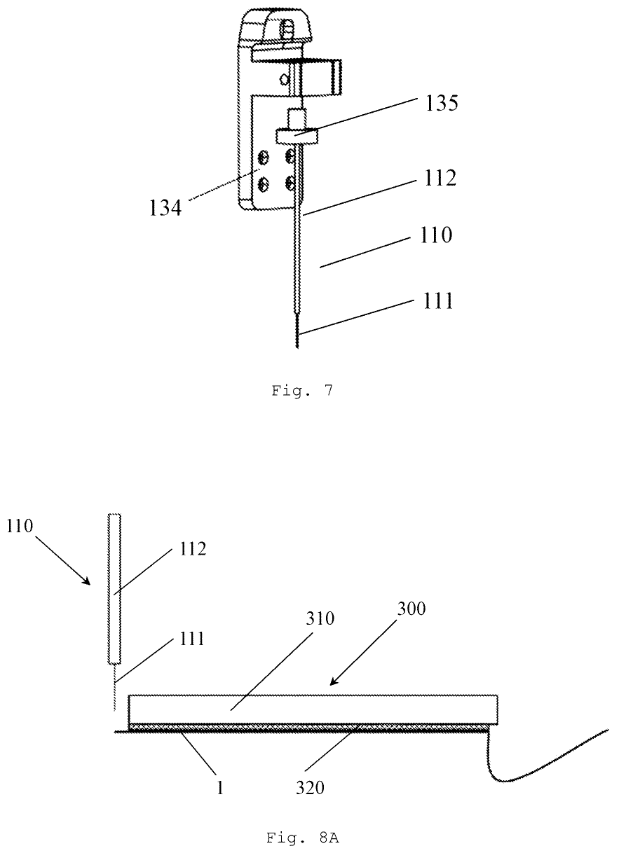

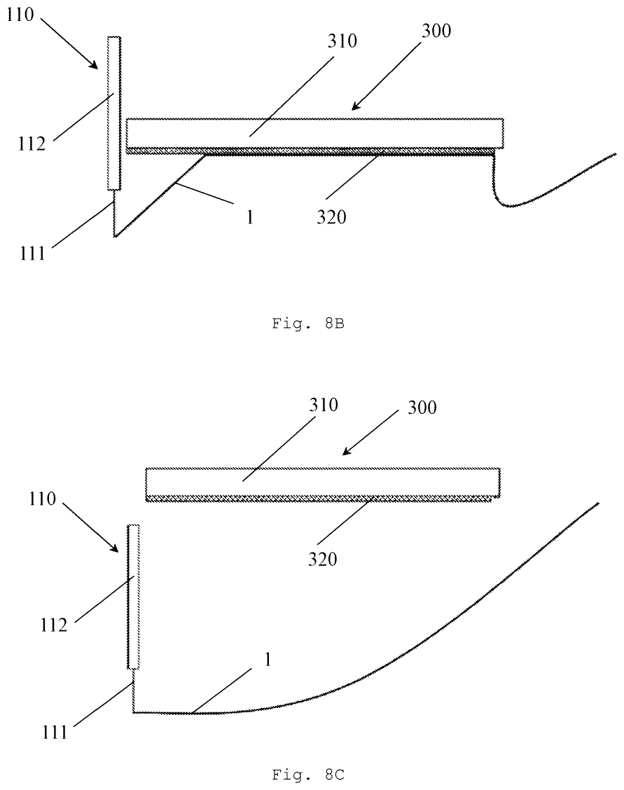

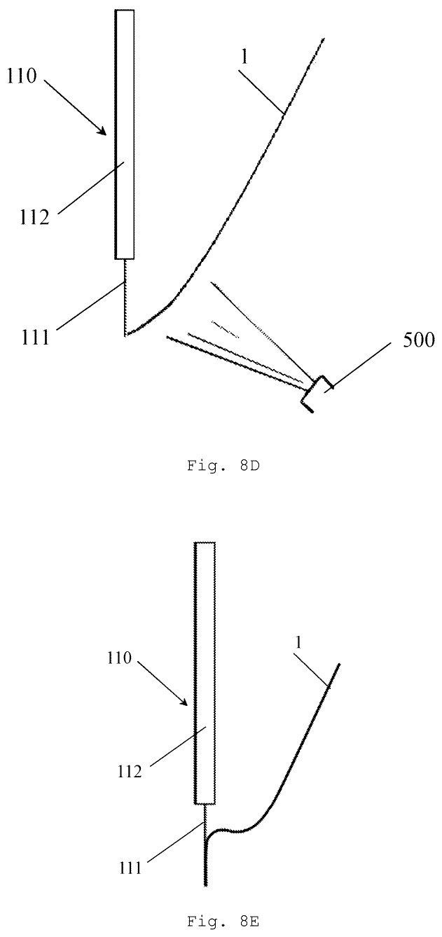

Referring herein to FIGS. 8A to 8C that describe a step of guiding the wire electrode in the method for operating the system of the present disclosure according to an embodiment of the present disclosure, it can be seen that the electrode fixation apparatus 300 includes a support plate 310 which may, on a surface thereof close to (or facing) the target object, detachably fix an electrode substrate 320. The wire electrode 1 may be adhered onto the electrode substrate 320 to be thereby fixed on the support plate 310. Due to high flexibility and low mechanical strength of the wire electrode 1, it's inconvenient to fix it on the support plate 310, and thus this can be aided by the electrode substrate 320 having a higher hardness than the wire electrode 1. The electrode substrate 320 may be made of high molecular polymer and manufactured together with the wire electrode 1, to provide limit and support for the wire electrode 1 before being implanted into the target object. In implementation, a plurality of wire electrodes 1 are generally formed together in batch, and such a plurality of wire electrodes 1 may be formed to adhere to the electrode substrate 320 side by side. According to the need of the implantation, one of the plurality of wire electrodes 1 may be implanted into the target object, or multiple of the plurality of wire electrodes 1 may be sequentially implanted into the target object.

The implantation subsystem may further include: an electrode position adjustment mechanism configured to adjust a position of the electrode fixation apparatus 300 along a plane perpendicular to the longitudinal direction, so that the guide apparatus is capable of being engaged with the wire electrode 1 fixed on the electrode fixation apparatus 300 during movement of the guide apparatus from a first side of the electrode fixation apparatus 300 away from the target object to a second side of the electrode fixation apparatus 300 close to the target object along the longitudinal direction. On a free end of the wire electrode 1, a ring, such as a circular ring, an elliptical ring, a semi-circular ring, or a rectangular ring, may be provided, through which a tip of the implantation needle 110 or guide apparatus can pass to be engaged with the free end of the wire electrode 1.

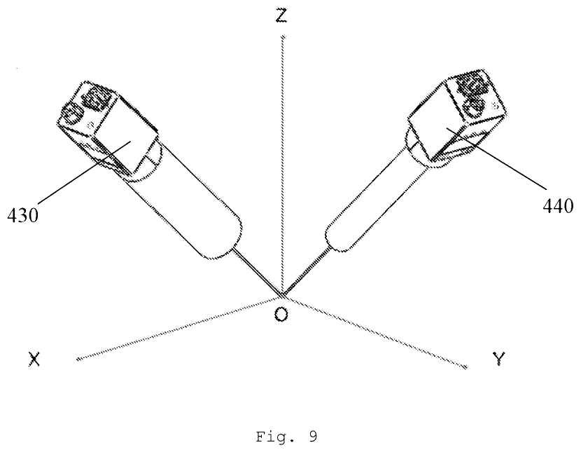

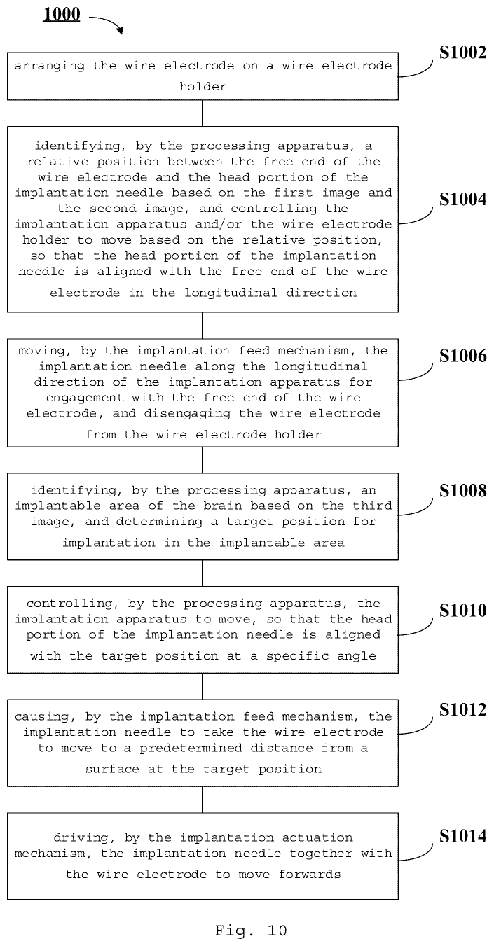

When the implantation needle 110 or the guide apparatus is engaged with the wire electrode 1, it is needed to observe and locate an engagement part at a front end of the wire electrode 1 and the tip of the implantation needle 110. As shown in FIGS. 1 to 3, for this purpose, the system for implanting the wire electrode 1 according to the embodiment of the present disclosure may further include: a first light source 410 for providing first light suitable for observing the implantation needle 110 and the free end of the wire electrode 1; a second light source 420 for providing second light suitable for observing the brain surface; a first camera 430 configured to acquire a first image of the head portion 111 and the free end of the wire electrode 1 with assistance of the first light; a second camera 440 configured to acquire a second image of the head portion 111 and the free end of the wire electrode 1 with assistance of the first light, wherein an optical path of the first camera 430 and an optical path of the second camera 440 are angled with respect to each other; a brain surface camera 450 configured to acquire a third image of the brain surface with assistance of the second light; and a processing apparatus configured to identify a relative position of the head portion 111 and the free end of the wire electrode 1 based on the first image and the second image, and identify an implantable area of the brain surface based on the third image. The first light includes one or more of white light, blue light, red light, infrared light, and near ultraviolet light, and/or the second light includes green light. The first camera 430 and the brain surface camera 450 may be arranged on the back plate 230. The second camera 440 may be arranged on a first carrying section of the carrying frame 240 that is opposite to and substantially parallel to the back plate 230. The first camera 430 and/or the second camera 440 are configured to be adjustable in position, so that a position thereof is adjusted to cause the head portion 111 and the free end of the wire electrode 1 to be presented in a field of view thereof with observable resolution. The first camera 430 and the second camera 440 may be arranged so that the optical path of the first camera 430 and the optical path of the second camera 440 meet at the free end of the wire electrode 1. As shown in FIG. 9, it is assumed that an observation target (the end of the wire electrode 1) is located at an origin 0 of a coordinate system in the figure. The first camera 430 and the second camera 440 need to observe the target simultaneously. The optical path of the first camera 430 and the optical path of the second camera 440 show observation directions of the two cameras. The first camera 430 and its lens may be arranged in an XZ plane, and the second camera 440 and its lens may be arranged in a YZ plane. An included angle between the optical path of the first camera 430 and an X-axis may be initially set to 45°, and may be adjusted according to requirements of a user. An included angle between the optical path of the second camera 440 and a Y-axis may also be initially set to 45°, and may be adjusted according to the requirements of the user.

In other words, the system for implanting the wire electrode 1 to the target object according to the embodiment of the present disclosure may further include an observation subsystem including: a vision module 400 configured to acquire a first image of an end of the guide apparatus that is used for engagement with the wire electrode 1 and an end of the wire electrode 1 fixed on the electrode fixation apparatus 300 that is used for engagement with the guide apparatus; and an operation control module configured to identify a relative position of the end of the guide apparatus and the end of the wire electrode 1 based on the first image, and control the electrode position adjustment mechanism to adjust the position of the electrode fixation apparatus 300 and/or control the transverse position adjustment mechanism to adjust the position of the actuation mechanism based on the identification result, so that the end of the guide apparatus is capable of be engaged with the end of the wire electrode 1 fixed on the electrode fixation apparatus 300 during the movement of the guide apparatus from the first side to the second side of the electrode fixation apparatus 300 along the longitudinal direction. The vision module 400 is further configured to acquire a second image of the target object, and the operation control module is further configured to identify the target implantation area of the target object based on the second image, and control the transverse position adjustment mechanism to adjust the position of the actuation mechanism based on the identification result, so that the guide apparatus is aligned with the target implantation area.

As shown in FIGS. 1 to 3, the system for implanting the wire electrode 1 may further include a spray apparatus 500 configured to keep the brain surface and/or a surrounding environment moist, and/or attach the wire electrode 1 to the implantation needle 110. In other words, the system for implanting the wire electrode 1 to the target object may further include an assistance subsystem including: a first spray apparatus arranged near the target object and configured to provide spray to keep a surface and/or a surrounding environment of the target object moist; and/or a second spray apparatus arranged near a position where the guide apparatus is engaged with the wire electrode 1 and configured to apply spray to the wire electrode 1 so that the wire electrode 1 is adhered to the guide apparatus.

FIGS. 8A to 8E are schematic views of steps of wire electrode guiding and spraying in a method for operating the system according to the present disclosure, according to an embodiment of the present disclosure. The method for operating the system according to the embodiment of the present disclosure is described below with reference to FIGS. 8A to 8C, wherein the method includes: arranging the wire electrode 1 on the wire electrode 1 holder; identifying, by the processing apparatus, a relative position between the free end of the wire electrode 1 and the head portion 111 of the implantation needle 110 based on the first image and the second image, and controlling the implantation apparatus 100 and/or the wire electrode 1 holder to move based on the relative position, so that the head portion 111 of the implantation needle 110 is aligned with the free end of the wire electrode 1 in the longitudinal direction; moving, by the implantation feed mechanism 120, the implantation needle 110 along the longitudinal direction of the implantation apparatus 100 for engagement with the free end of the wire electrode 1, and disengaging the wire electrode 1 from the wire electrode 1 holder; identifying, by the processing apparatus, an implantable area of the brain based on the third image, and determining a target position for implantation in the implantable area; controlling, by the processing apparatus, the implantation apparatus 100 to move, so that the head portion 111 of the implantation needle 110 is aligned with the target position implantation feed mechanism 120 at a specific angle; causing, by the implantation feed mechanism 120 implantation feed mechanism 120, the implantation needle 110 to take the wire electrode 1 to move to a predetermined distance from a surface at the target position; and driving, by the implantation actuation mechanism 130, the implantation needle 110 together with the wire electrode 1 to move forwards. After the implantation operation of the implantation needle 110 is completed, the implantation needle 110 may be driven by the implantation actuation mechanism 130 to retract. The arranging the wire electrode 1 on the wire electrode 1 holder includes: arranging a head section of the wire electrode 1 on the wire electrode 1 holder, and making the head section of the wire electrode 1 protrude from the wire electrode 1 holder so that the head section becomes the free end; and suspending a section between a tail section for circuit connection and the head section of the wire electrode 1 so that the section is in a non-tensioned state.

As shown in FIG. 8A, a front section of the wire electrode 1 is arranged on the electrode fixation apparatus 300, and an engagement part of the wire electrode 1 that is located at the front end protrudes from the electrode fixation apparatus 300, so as to be engaged with the guide apparatus. After the wire electrode 1 is fixed on the electrode fixation apparatus 300, there is a section (referred to as a “posterior section” hereinafter) between a rear section for circuit connection and the front section of the wire electrode 1, which is suspended to be in a non-tensioned state (a “suspending part” as shown in the figure).

The actuation mechanism may be positioned substantially on an extension line from the target object for implantation to the electrode fixation apparatus 300 and drives the guide apparatus for guiding the wire electrode 1 to move substantially towards the target object. As shown, the guide apparatus may include the head portion 111 located at an end of the guide apparatus and the tube portion 112 for mounting the head portion 111, and the head portion 111 may be mounted on the tube portion 112 by, for example, an adhesive, a ferrule, or the like. The actuation mechanism may be positioned at a top of the tube portion 112, thereby driving the head portion 111 via driving the tube portion 112 to move. After the guide apparatus is engaged with the engagement part of the wire electrode 1 fixed on the electrode fixation apparatus 300, the actuation mechanism drives the guide apparatus to continue moving, e.g., downwards, to pull the wire electrode 1, i.e., to apply a pull force to the front end of the wire electrode 1, thereby at least partially separating the wire electrode 1 from the electrode fixation apparatus 300, as shown in FIG. 8B. The guide apparatus can continue moving downwards towards the target object under the driving of the actuation mechanism, to continue applying a pull force to the wire electrode 1 via the front end of the wire electrode 1, so that the wire electrode 1 can be completely disengaged from the electrode fixation apparatus 300, as shown in FIG. 8C. Since the posterior section of the wire electrode 1 is suspended (not tightly fixed on the electrode fixation apparatus 300) and is in a non-tensioned state, the part between the front end of the wire electrode 1 and the rear end thereof (not shown, referring to an end for circuit connection) is also suspended in a non-tensioned state after the wire electrode 1 is completely disengaged from the electrode fixation apparatus 300.

After the implantation needle 110 is engaged with the free end of the wire electrode 1, spray is applied to the wire electrode 1 suspended beside the implantation needle 110 by the spray apparatus 500, so that the head section of the wire electrode 1 is attached on the implantation needle 110.

The spray apparatus 500 may be positioned to face a part of the wire electrode 1 that is separated from the electrode fixation apparatus 300 (especially facing a section close to the front end) and face the guide apparatus, and the spray apparatus 500 may be positioned to be capable of spraying liquid with a spray force directed substantially from the section of the wire electrode 1 that is close to the front end to the guide apparatus, as shown in FIG. 8D, so that at least the section of the wire electrode 1 that is close to the front end is attached to the guide apparatus under the action of the sprayed liquid, as shown in FIG. 8E. The spray apparatus 500 allows the liquid to be sprayed in an atomized state. The sprayed liquid may include pure water or a solution suitable for the target object.

The spray apparatus 500 is provided so that at least the section of the wire electrode 1 that is close to the front end is attached to the guide apparatus, which can avoid formation of large incision damage to biological tissue of the target object. Furthermore, further technical effects can be brought by spraying the liquid. After the guide apparatus is engaged with the engagement part of the wire electrode 1 fixed on the electrode fixation apparatus 300 and the guide apparatus causes the wire electrode 1 to be completely disengaged from the electrode fixation apparatus 300, as shown in FIG. 8C, under gravity of the wire electrode 1 itself, its engagement part may be disengaged from the guide apparatus, resulting in undesired separation of the wire electrode 1 from the guide apparatus. However, after the wire electrode 1 is attached to the guide apparatus by spraying the liquid, that is, the wire electrode 1 is made to be in a state shown in FIG. 8E, the engagement between the wire electrode 1 and the guide apparatus can be strengthened, avoiding that the wire electrode 1 is disengaged from the guide apparatus due to the gravity of the electrode itself.

In this embodiment, controlling the implantation apparatus 100 to move includes: sliding the implantation apparatus 100 on the arc-shaped rail 220 and rotating the arc-shaped rail 220 about a rotation axis normal to an arc of the arc-shaped rail 220 and extending through a center of the arc-shaped rail 220, and/or moving the robotic arm 210 that is freely movable in space, to change an orientation of the head portion 111 of the implantation needle 110.

When the implantation apparatus 100 and the wire electrode 1 are observed, an illumination angle, illumination intensity and/or light source color of the first light source 410 can be adjusted, and imaging parameters, a position and/or optical path direction of the first camera 430 and/or the second camera 440 can be adjusted, so that the first camera 430 and the second camera 440 are capable of obtaining respective clear images. Furthermore, an illumination angle, illumination intensity and/or light source color of the second light source 420 can be adjusted, and imaging parameters, a position and/or optical path direction of the brain surface camera 450 can be adjusted, so that the brain surface camera 450 is capable of obtaining a clear image.

When the implantation apparatus 100 is controlled by the processing apparatus to move, the implantation apparatus 100 may be controlled to move along the longitudinal direction until the tip of the head portion 111 is in contact with the brain surface, and a position of the tip of the head portion 111 is marked in the third image as an initial position for subsequently controlling the implantation apparatus 100 to move so that the head portion 111 of the implantation needle 110 is aligned with the target position. In addition, it is possible to control the implantation apparatus 100 to move along the longitudinal direction until the tip of the head portion 111 is in contact with the brain surface, and then control the implantation apparatus 100 to retract by a predetermined distance in the longitudinal direction, so that the implantation needle 110 takes the wire electrode 1 to move to the predetermined distance from the surface at the target position. To determine that the tip of the head portion 111 has been in contact with the brain surface, the contact of the tip of the head portion 111 with the surface at the target position may be detected by using a detection circuit that is closed upon the tip of the head portion 111 contacting the surface at the target position. Here, an XY position of the head portion 111 can be fine-tuned by using a micro motor on the head portion 111 to align a drop point with a point in the implantable area. After the alignment with the implantation point, the implantation feed mechanism 120 in the implantation apparatus 100 slowly moves the head portion 111 towards the target position, retracts the implantation needle 110 to a predetermined height after the head portion 111 is in contact with the brain surface, and then drives the implantation actuation mechanism 130 to rapidly implant the implantation needle 110 downwards, wherein the predetermined height is related to an implantation depth and a defined stroke of the implantation actuation mechanism 130 (for example, when the implantation depth is 2 mm and the defined stroke of the implantation actuation mechanism 130 is 5 mm, the implantation needle 110 is lifted to a position where the tip thereof is 3 mm from the brain surface). Then, the implantation needle 110 is retracted rapidly, for example, at a speed of 30 m²/s, so that the wire electrode 1 is disengaged from the implantation needle 110 and left within the brain tissue. After the implantation needle 110 is retracted, the head portion 111 of the implantation needle 110 may be cleaned. Then, the electrode fixation apparatus 300 may be moved for next implantation.

Although some specific embodiments of the present disclosure have been described in detail by way of examples, it should be understood by those skilled in the art that the above examples are for illustration only and are not intended to limit the scope of the present disclosure. The embodiments disclosed herein may be combined arbitrarily without departing from the spirit and scope of the present disclosure. Those skilled in the art will also appreciate that various modifications may be made to the embodiments without departing from the scope and spirit of the present disclosure. The scope of the present disclosure is defined by the appended claims.

What is claimed is:

1. A system for implanting a wire electrode, the system configured for implanting the wire electrode into a brain of an organism, the system comprising:
an implantation apparatus comprising an implantation needle configured to engage, with a head portion thereof, a free end of the wire electrode so as to take the wire electrode to move, an implantation feed mechanism configured to move the implantation needle along a longitudinal direction of the implantation apparatus, and an implantation actuation mechanism configured to drive the implantation needle so that the head portion of the implantation needle penetrates into the brain;
a first light source for providing first light suitable for observing the implantation needle and the free end of the wire electrode;
a second light source for providing second light suitable for observing a surface of the brain;
a first camera configured to acquire a first image of the head portion and the free end of the wire electrode with assistance of the first light;
a second camera configured to acquire a second image of the head portion and the free end of the wire electrode with assistance of the first light, wherein an optical path of the first camera and an optical path of the second camera are angled with respect to each other;
a brain surface camera configured to acquire a third image of the surface of the brain with assistance of the second light; and
a processing apparatus configured to identify a relative position of the head portion and the free end of the wire electrode based on the first image and the second image, and identify an implantable area of the surface of the brain based on the third image,
wherein the implantation apparatus is provided with an implantation movement mechanism configured to enable the implantation apparatus to implant the wire electrode at different angles in different orientations.

2. The system according to claim 1, wherein the implantation movement mechanism comprises a robotic arm that is freely movable in space.

3. The system according to claim 1, wherein the implantation movement mechanism comprises an arc-shaped rail on which the implantation apparatus is slidable.

4. The system according to claim 3, wherein the arc-shaped rail is rotatable about a rotation axis normal to an arc of the arc-shaped rail and extending through a center of the arc-shaped rail.

5. The system according to claim 3, wherein the implantation movement mechanism comprises a robotic arm that is freely movable in space, the implantation apparatus being connectable with the robotic arm via the arc-shaped rail.

6. The system according to any of claims 1 to 5, wherein the implantation apparatus further comprises a first implantation carrier for arranging the implantation actuation mechanism, the first implantation carrier being capable of performing rectilinear motion by the implantation feed mechanism.

7. The system according to claim 6, wherein the implantation feed mechanism comprises a first guide block and a first rectilinear rail, the first guide block being capable of performing rectilinear motion on the first rectilinear rail, wherein the first implantation carrier is integrally configured with or detachably arranged on the first guide block of the implantation feed mechanism.

8. The system according to claim 7, wherein the first guide block is capable of performing rectilinear motion on the first rectilinear rail via a drive mechanism which is capable of transforming rotational motion into the rectilinear motion of the first guide block.

9. The system according to claim 6, wherein the implantation apparatus further comprises a second implantation carrier for fixing the implantation needle, the implantation actuation mechanism being capable of driving the second implantation carrier to perform rectilinear motion, so that the head portion of the implantation needle penetrates into the brain.

10. The system according to claim 9, wherein the second implantation carrier comprises an implantation needle fixation block, the implantation needle being capable of being detachably fixed on the implantation needle fixation block.

11. The system according to claim 10, wherein the implantation needle is capable of being fixed on the implantation needle fixation block via an adapter.

12. The system according to claim 11, wherein the implantation needle is connected on the adapter in a manner of material locking.

13. The system according to claim 9, wherein the second implantation carrier is provided with a second guide block and a second rectilinear rail, the second guide block being capable of performing rectilinear motion on the second rectilinear rail, the second implantation carrier being arranged on the second guide block, so that the second implantation carrier, when driven by the implantation actuation mechanism, performs rectilinear motion on the second rectilinear rail via the second guide block in a guided manner.

14. The system according to claim 13, wherein the implantation actuation mechanism is configured as an electromagnetic actuation mechanism which is capable of driving the second implantation carrier together with the second guide block to move on the second rectilinear rail for a defined stroke, so that the head portion is moved for the defined stroke.

15. The system according to claim 13, wherein the implantation actuation mechanism is configured as a pneumatic actuation mechanism, with a stopper provided on the second rectilinear rail, the pneumatic actuation mechanism being capable of driving the second implantation carrier together with the second guide block to move on the second rectilinear rail until the second guide block is stopped at the stopper, so that the head portion is moved for a defined stroke.

16. The system according to any of claims 1 to 5, wherein the implantation needle is configured in two pieces, the implantation needle comprising a tube portion and the head portion, the head portion being fixed on an end of the tube portion or embedded into the tube portion.

17. The system according to claim 2 or 5, wherein a back plate is arranged on a free end of the robotic arm, wherein the implantation apparatus is arranged on the back plate.

18. The system according to claim 17, wherein the first camera and the brain surface camera are arranged on the back plate.

19. The system according to claim 17, wherein the system comprises a wire electrode holder which is capable of holding the wire electrode and is arranged on a carrying frame fixed on the back plate.

20. The system according to claim 19, wherein the wire electrode holder is movably arranged on the carrying frame.

21. The system according to claim 19, wherein the wire electrode holder is connected to the carrying frame by a plane movement mechanism, the plane movement mechanism being configured to move along both a direction substantially parallel to the back plate and a direction substantially perpendicular to the back plate.

22. The system according to claim 19, wherein the second camera is arranged on a first carrying section of the carrying frame that is opposite and substantially parallel to the back plate.

23. The system according to any of claims 1 to 5, wherein the first camera and/or the second camera is configured to be adjustable in position, so that a position thereof is adjusted such that the head portion and the free end of the wire electrode are presented in a field of view thereof with observable resolution.

24. The system according to any of claims 1 to 5, wherein the first camera and the second camera are arranged so that the optical path of the first camera and the optical path of the second camera meet at the free end of the wire electrode.

25. The system according to any of claims 1 to 5, wherein a ring is provided on the free end of the wire electrode, through which the head portion is capable of passing to be engaged with the free end of the wire electrode.

26. The system according to any of claims 1 to 5, wherein the first light comprises one or more of white light, blue light, red light, infrared light, and near ultraviolet light, and/or the second light comprises green light.

27. The system according to any of claims 1 to 5, wherein the head portion of the implantation needle is provided with a micro motor which is capable of moving the head portion in a transverse direction of the implantation apparatus.

28. The system according to any of claims 1 to 5, wherein the system further comprises a spray apparatus configured to keep the surface and/or a surrounding environment of the brain moist, and/or attach the wire electrode to the implantation needle.

29. The system according to any of claims 1 to 5, wherein the implantation actuation mechanism is capable of causing the head portion to penetrate into the brain at a speed between 0.5 m/s and 5 m/s or between 0.01 m/s and 10 m/s.

30. The system according to any of claims 1 to 5, wherein the system further comprises a brain surface detection apparatus which is capable of detecting that the conductive head portion is in contact with the surface of the brain.

31. The system according to claim 30, wherein the brain surface detection apparatus comprises a detection circuit, with the head portion connected on the detection circuit and another part of the implanted organism connected to the circuit, wherein a voltage measurement apparatus is connected on the circuit, so that when the head portion is in contact with the surface of the brain, the circuit is closed and the voltage measurement apparatus measures a voltage to indicate the head portion contacting the surface of the brain.

32. A system for implanting a bioelectrode to a target object, comprising:
an actuation mechanism configured to drive a guide apparatus engaged with the bioelectrode to move, so that the guide apparatus, together with the bioelectrode, enters the target object;
a position adjustment mechanism configured to adjust a position of the actuation mechanism, so that the guide apparatus approaches a target implantation area of the target object; and
an orientation adjustment mechanism configured to adjust an orientation of the actuation mechanism, so that the guide apparatus is capable of entering, at the target implantation area, the target object at a specific angle with respect to a surface of the target implantation area.

33. The system according to claim 32, wherein the orientation adjustment mechanism is implemented as a robotic arm.

34. The system according to claim 32 or 33, wherein at least a part of the position adjustment mechanism is implemented as a robotic arm.

35. The system according to claim 32, wherein the position adjustment mechanism comprises a coarse position adjustment module and a fine position adjustment module, wherein at least a part of the coarse position adjustment module and the orientation adjustment mechanism is implemented as a robotic arm, and the fine position adjustment module comprises a micro motor.

36. The system according to claim 32, wherein the orientation adjustment mechanism comprises an arc-shaped rail, a first end of the actuation mechanism away from the guide apparatus being mounted to the arc-shaped rail, so that the orientation adjustment mechanism adjusts the orientation of the actuation mechanism.

37. The system according to claim 36, wherein the arc-shaped rail is configured to be rotatable about an axis normal to an arc of the arc-shaped rail and extending through a center of the arc-shaped rail.

38. The system according to claim 36, wherein the orientation adjustment mechanism is provided on the position adjustment mechanism, so that the position adjustment mechanism adjusts the position of the actuation mechanism by adjusting a position of the orientation adjustment mechanism.

39. The system according to claim 32 or 38, wherein the position adjustment mechanism comprises a longitudinal adjustment module and a transverse adjustment module, wherein
the longitudinal adjustment module is configured to adjust the position of the actuation mechanism in a longitudinal direction of the actuation mechanism, so that the guide apparatus approaches the target implantation area along the longitudinal direction; and
the transverse adjustment module is configured to adjust the position of the actuation mechanism in a plane perpendicular to the longitudinal direction, so that the guide apparatus approaches the target implantation area along a transverse direction.

40. The system according to claim 32, wherein the target object comprises a brain.

41. The system according to claim 32, wherein the bioelectrode comprises a wire-like flexible electrode.

42. A system for implanting a wire electrode to a target object, the system comprising an implantation subsystem, the implantation subsystem comprising:
an actuation mechanism configured to drive a guide apparatus engaged with the wire electrode to move in a direction of approaching the target object at a first speed along a longitudinal direction of the actuation mechanism, so that the guide apparatus, together with the wire electrode, enters the target object; and
a longitudinal position adjustment mechanism configured to adjust a position of the actuation mechanism at a second speed along the longitudinal direction, so that the guide apparatus approaches a target implantation area of the target object along the longitudinal direction,
wherein the first speed is greater than the second speed.

43. The system according to claim 42, wherein the actuation mechanism is further configured to drive the guide apparatus that enters the target object to retract in a direction away from the target object at a third speed along the longitudinal direction, so that the guide apparatus exits from the target object.

44. The system according to claim 43, wherein the first speed and the third speed are both between 0.5 m/s and 5 m/s, or between 0.01 m/s and 10 m/s.

45. The system according to claim 43, wherein an acceleration of the retraction of the guide apparatus driven by the actuation mechanism is between 25 m/s² and 35 m/s².

46. The system according to claim 42 or 43, wherein the actuation mechanism comprises an electromagnetic actuation module, a high-speed motor actuation module, or a pneumatic module.

47. The system according to claim 42 or 43, wherein the actuation mechanism further configured to drive the guide apparatus to move for a specific stroke along the longitudinal direction, and the implantation subsystem further comprises:
an implantation depth control module configured to control the longitudinal position adjustment mechanism, so that a tip of the guide apparatus is located at a predetermined distance from a surface of the target implantation area, and then control the actuation mechanism to drive the guide apparatus to move.

48. The system according to claim 47, wherein the implantation depth control module is further configured to control the longitudinal position adjustment mechanism, so that the guide apparatus approaches the target implantation area until the tip of the guide apparatus is in contact with the surface of the target implantation area, and then control the longitudinal position adjustment mechanism, so that the guide apparatus retracts by the predetermined distance, and the tip of the guide apparatus is located at the predetermined distance from the surface of the target implantation area.

49. The system according to claim 48, wherein the implantation depth control module detects that the tip of the guide apparatus is in contact with the surface of the target implantation area by a detection circuit that is closed upon the tip of the guide apparatus contacting the surface of the target implantation area.

50. The system according to claim 42 or 43, wherein the implantation subsystem further comprises:
an orientation adjustment mechanism configured to adjust an orientation of the actuation mechanism, so that the guide apparatus is capable of entering, at the target implantation area of the target object, the target object at a specific angle with respect to a surface of the target implantation area.

51. The system according to claim 42 or 43, wherein the implantation subsystem further comprises:
a transverse position adjustment mechanism configured to adjust the position of the actuation mechanism in a plane perpendicular to the longitudinal direction, so that the guide apparatus is aligned with the target implantation area.

52. The system according to claim 51, wherein the implantation subsystem further comprises:
an electrode fixation apparatus configured to detachably fix, on a surface thereof close to the target object, the wire electrode.

53. The system according to claim 52, wherein the electrode fixation apparatus is configured to detachably fix, on the surface thereof close to the target object, an electrode substrate, wherein the wire electrode is adhered onto the electrode substrate and thus fixed on the electrode fixation apparatus.

54. The system according to claim 53, wherein the electrode substrate has a higher hardness than the wire electrode.

55. The system according to claim 53, wherein there are a plurality of wire electrodes to be implanted, the plurality of wire electrodes being adhered side by side onto the electrode substrate.

56. The system according to claim 52, wherein the implantation subsystem further comprises:
an electrode position adjustment mechanism configured to adjust a position of the electrode fixation apparatus along a plane perpendicular to the longitudinal direction, so that the guide apparatus is capable of be engaged with the wire electrode fixed on the electrode fixation apparatus during movement of the guide apparatus from a first side of the electrode fixation apparatus away from the target object to a second side of the electrode fixation apparatus close to the target object along the longitudinal direction.

57. The system according to claim 56, further comprising an observation subsystem comprising:
a vision module configured to acquire a first image of an end of the guide apparatus that is used for engagement with the wire electrode and an end of the wire electrode fixed on the electrode fixation apparatus that is used for engagement with the guide apparatus; and
an operation control module configured to identify a relative position of the end of the guide apparatus and the end of the wire electrode based on the first image, and control the electrode position adjustment mechanism to adjust the position of the electrode fixation apparatus and/or control the transverse position adjustment mechanism to adjust the position of the actuation mechanism based on the identification result, so that the end of the guide apparatus is capable of be engaged with the end of the wire electrode fixed on the electrode fixation apparatus during the movement of the guide apparatus from the first side to the second side of the electrode fixation apparatus along the longitudinal direction.

58. The system according to claim 57, wherein
the vision module is further configured to acquire a second image of the target object, and
the operation control module is further configured to identify the target implantation area of the target object based on the second image, and control the transverse position adjustment mechanism to adjust the position of the actuation mechanism based on the identification result, so that the guide apparatus is aligned with the target implantation area.

59. The system according to claim 42, further comprising an assistance subsystem comprising:
a first spray apparatus arranged near the target object and configured to provide spray to keep a surface and/or a surrounding environment of the target object moist; and/or
a second spray apparatus arranged near a position where the guide apparatus is engaged with the wire electrode and configured to apply spray to the wire electrode, so that the wire electrode is attached to the guide apparatus.

60. The system according to claim 42, wherein the target object comprises a non-flat surface.

61. The system according to claim 42, wherein the transverse position adjustment mechanism has an adjustment accuracy of less than 4 μm.

62. A method for operating the system according to any of claims 1 to 31, comprising:
arranging the wire electrode on a wire electrode holder;
identifying, by the processing apparatus, a relative position between the free end of the wire electrode and the head portion of the implantation needle based on the first image and the second image, and controlling the implantation apparatus and/or the wire electrode holder to move based on the relative position, so that the head portion of the implantation needle is aligned with the free end of the wire electrode along the longitudinal direction;
moving, by the implantation feed mechanism, the implantation needle along the longitudinal direction of the implantation apparatus for engagement with the free end of the wire electrode, and disengaging the wire electrode from the wire electrode holder;
identifying, by the processing apparatus, an implantable area of the brain based on the third image, and determining a target position for implantation in the implantable area;
controlling, by the processing apparatus, the implantation apparatus to move, so that the head portion of the implantation needle is aligned with the target position implantation feed mechanism at a specific angle;
causing, by the implantation feed mechanism implantation feed mechanism, the implantation needle to take the wire electrode to move to a predetermined distance from a surface at the target position; and
driving, by the implantation actuation mechanism, the implantation needle together with the wire electrode to move forwards.

63. The method according to claim 62, further comprising:
driving, by the implantation actuation mechanism, the implantation needle to retract.

64. The method according to claim 62, wherein, the arranging the wire electrode on a wire electrode holder comprises:
arranging a head section of the wire electrode on the wire electrode holder, and making a head end of the wire electrode protrude from the wire electrode holder so that the head end becomes the free end; and
suspending a section between a tail section for circuit connection and the head section of the wire electrode so that the section is in a non-tensioned state.

65. The method according to claim 64, further comprising:
after the implantation needle is engaged with the free end of the wire electrode, applying, by a spray apparatus, spray to the wire electrode suspended beside the implantation needle, so that the head section of the wire electrode is attached on the implantation needle.

66. The method according to claim 62, wherein the controlling the implantation apparatus to move comprises: sliding the implantation apparatus on an arc-shaped rail and rotating the arc-shaped rail about a rotation axis normal to an arc of the arc-shaped rail and extending through a center of the arc-shaped rail, and/or moving a robotic arm that is freely movable in space, to change an orientation of the head portion of the implantation needle.

67. The method according to claim 62, further comprising:
adjusting an illumination angle, illumination intensity and/or light source color of the first light source, and adjusting imaging parameters, a position and/or optical path direction of the first camera and/or the second camera, so that the first camera and the second camera are capable of obtaining respective clear images.

68. The method according to claim 62, further comprising:
adjusting an illumination angle, illumination intensity and/or light source color of the second light source, and adjusting imaging parameters, a position and/or optical path direction of the brain surface camera, so that the brain surface camera is capable of obtaining a clear image.

69. The method according to claim 62, further comprising:
controlling the implantation apparatus to move along the longitudinal direction until a tip of the head portion is in contact with the surface of the brain, and marking, in the third image, a position of the tip of the head portion as an initial position for subsequently controlling the implantation apparatus to move so that the head portion of the implantation needle is aligned with the target position.

70. The method according to claim 62, further comprising:
controlling the implantation apparatus to move along the longitudinal direction until a tip of the head portion is in contact with the surface of the brain, and then controlling the implantation apparatus to retract by the predetermined distance along the longitudinal direction, so that the implantation needle takes the wire electrode to move to the predetermined distance from the surface at the target position.

71. The method according to claim 69 or 70, wherein the contact of the tip of the head portion with the surface at the target position is detected by using a detection circuit that is closed upon the tip of the head portion contacting the surface at the target position.

72. The method according to claim 63, further comprising:
after the implantation needle is retracted, cleaning the head portion of the implantation needle.
